(19) United States
(12) Patent Application Publication
(10) Pub. No.: US 2026/0314596 A1
(43) Pub. Date: Oct. 15, 2026

(54) METHOD FOR INHIBITING WATER EVAPORATION AND AN APPLICATION OF AN ANTI-EVAPORATION AGENT

(71) Applicant: CHENGDU HANOVA BIOSCIENCES CO., LTD, Chengdu (CN)

(72) Inventors: Tingzhuo CHEN, Chengdu (CN); Jiayu LIU, Chengdu (CN); Quan SHI, Chengdu (CN)

(21) Appl. No.: 19/208,092

(22) Filed: May 14, 2025

(30) Foreign Application Priority Data
Apr. 10, 2025 (CN) 202510450020.7

(51) Int. Cl. A01N 41/02; A01N 25/02; A01N 25/30; A01P 21/00

(52) U.S. Cl. CPC A01N 41/02; A01N 25/02; A01N 25/30; A01P 21/00

(57) ABSTRACT

The present invention relates to the technical field of agricultural chemistry, in particular to a method for inhibiting water evaporation and an application of an anti-evaporation agent; by applying the sulfite ester compounds, the water evaporation rate is significantly reduced, which is adaptable to the application methods such as foliar spraying, and trickle irrigation, etc. The method of the present invention has the advantages of easy operation, environmental friendliness, and controllable cost, and is suitable for agricultural water-saving and precision pesticide application in arid regions.

### TECHNICAL FIELD

The present invention relates to the technical field of agricultural chemistry, in particular to a method for inhibiting water evaporation and the application of an anti-evaporation agent.

### BACKGROUND

Global agriculture is facing increasingly severe dual challenges of water resources scarcity and climate change. According to statistics from the Food and Agriculture Organization of the United Nations, agricultural water accounts for 70% of global freshwater consumption, and the evaporation loss rate of water under traditional irrigation modes is up to 50%-70%, exacerbating the regional water resource crises. In the context of climate change, extreme high temperatures and drought events occur frequently, further threatening water balance and yield stability of crops. How to improve water use efficiency through technological innovations has become a core proposition for ensuring food security and sustainable agricultural development.

In the attempt to inhibit water evaporation, there have been reports on the use of anti-evaporation additives.

Anti-evaporation additives, as a type of substances that reduce the water evaporation rate through physical or chemical mechanisms, have received widespread attention in recent years. Its mechanism of action mainly includes but is not limited to forming hydrophobic film layers on soil and leaf surfaces, regulating plant stomatal behaviors, or enhancing the water absorption capacity of the root system, etc. At the same time, improving the droplet anti-evaporation property can prevent the rapid evaporation of water from the medicinal solution deposited and adsorbed on the leaves. If the water evaporates completely before the active ingredients are fully absorbed and adsorbed by the leaves or targets, the absorption and utilization of the active ingredients will be affected, thereby affecting the pesticide effect. Existing research have shown that anti-evaporation agents can reduce soil water evaporation by 30%-60%, significantly prolonging the irrigation interval periods, and especially showing outstanding water-saving and yield-increasing effects in arid and semi-arid regions.

However, different anti-evaporation additives also have corresponding limitations. Currently, the anti-evaporation additives mainly include four categories such as vegetable oils, mineral oils, silicone, and surfactants, and their performance bottlenecks and environmental risks have become key obstacles to the precise and efficient utilization of pesticides. Vegetable oil-based additives are prone to oxidation and decomposition under light exposure and have high environmental sensitivity. They are prone to separation from medicinal solutions under drought conditions and may cause crop pesticide damage; mineral oil based additives may cause an increase in droplet size due to their high viscosity, affecting their penetration, and they are difficult to biodegrade, leading to soil pollution; although the silicone based additives can significantly reduce surface tension, their high volatility leads to rapid droplet shrinkage, and too small droplets can easily cause drift risks; and the surfactant based additives have the problems of biotoxicity and strong soil adsorption, and some synthetic components also have ecological hazards.

The carbamate-based inhibitor developed by Wu Yan's team has an evaporation inhibition rate of 30-40% in an environment above 30° C., but its high temperature tolerance and efficacy still need to be improved (Wu Yan, Fang Xiting, Yang Fangming. Preparation and Performance of Carbamate-based Water Evaporation Inhibitors [J]. Journal of Tianjing University of Science & Technology, 2014, 29 (03): 34-39); Ma Yan et al., used a combination of octadecanol and n-propanol and found that an increase in surface tension led to a decrease in spreading rate, and the effective duration was short (Ma Yan, Ye Hanchun, Lv Xifeng, et al. Comparative Experiment and Study on Homogeneous/Heterogeneous Water Evaporation Retardant [J]. Yellow River, 2015, 37 (6)); the patent (CN106417262A) discloses a chitosan based sol-gel system, the evaporation inhibition rate of which decreases significantly and the thermal stability is insufficient at high temperature above 36° C.; in the patent (CN101235267B), the long chain fatty alcohol (such as hexadecanol) and short chain alcohol (such as n-butanol) are compounded to form a heterogeneous W/O type emulsion, which spreads on the water surface to form a molecular film to inhibit evaporation, and the inhibition effect is good, but the oil phase solvent is petroleum ether, the long-term use of which may cause potential pollution to the aquatic ecosystem, and the AEO emulsifiers have poor biodegradability and may accumulate in water, posing a threat to the safety of aquatic organisms.

Therefore, it is particularly important to constantly discover or provide new products with high safety and good water evaporation inhibition effects.

Sulfite ester compounds are currently used to kill agricultural mites. For example, CN 118373760 A has disclosed multiple structurally improved sulfite ester compounds, which exhibit better mite killing effects than Propargite. However, there are currently no reports about using such compounds to inhibit water evaporation.

### SUMMARY

The present invention first discovered the effect of sulfite ester compounds on inhibiting water evaporation, expanding the application scope of sulfite ester compounds and providing a new option for anti-evaporation agents in agriculture.

The present invention provides a method for inhibiting water evaporation, wherein, applying sulfite ester compounds to water, the structural formula of the sulfite ester compounds is as follows:

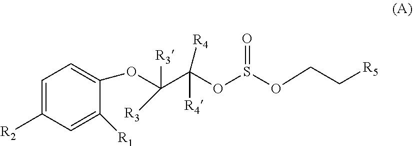

Wherein, R₁, and R₂ are selected from the group consisting of halogen;
R₃, R₃′, R₄, and R₄′ are respectively independently selected from the group consisting of hydrogen, C₁-C₅ alkyl, and C₁-C₅ alkenyl; and
R₅ is selected from a group consisting of halogen.

In some specific embodiments of the present invention, the R₁, and R₂ are selected from the group consisting of Cl; and R₅ is selected from the group consisting of F.

In some specific embodiments of the present invention, R₃, and R₃′ are selected from the group consisting of hydrogen; and R₄, and R₄′ are respectively independently selected from the group consisting of hydrogen, —CH₃, —CH₂CH₃, and —CH═CH₂.

In some specific embodiments of the present invention, the sulfite ester compound is selected from one of the following compounds or a combination of more than two of them:

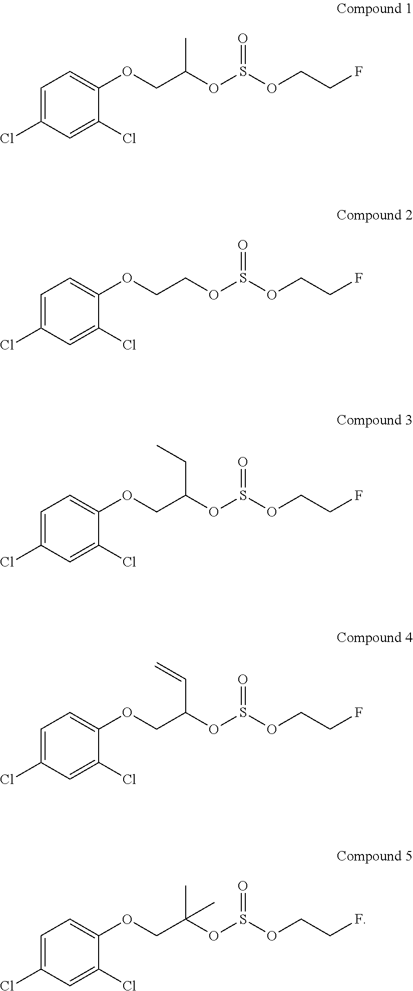 [SMILES: CC(COc1ccc(Cl)cc1Cl)OS(=O)OCCF ; O=S(OCCF)OCCOc1ccc(Cl)cc1Cl ; CCC(COc1ccc(Cl)cc1Cl)OS(=O)OCCF ; C=CC(COc1ccc(Cl)cc1Cl)OS(=O)OCCF ; CC(C)(COc1ccc(Cl)cc1Cl)OS(=O)OCCF]

In some specific embodiments of the present invention, the sulfite ester compound is selected from one of the following compounds or a combination of more than two of them:

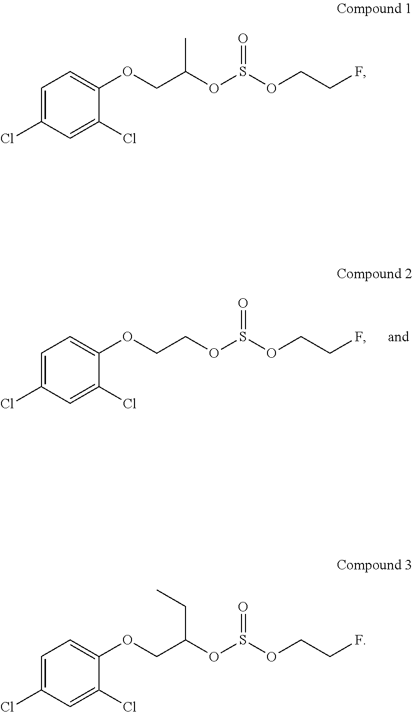 [SMILES: CC(COc1ccc(Cl)cc1Cl)OS(=O)OCCF ; O=S(OCCF)OCCOc1ccc(Cl)cc1Cl ; CCC(COc1ccc(Cl)cc1Cl)OS(=O)OCCF]

In the present invention, the sulfite ester compounds exhibit significant water evaporation inhibition effect over a wide temperature range (20° C.-50° C.), and is especially suitable for high-temperature and arid environments.

In the present invention, when the sulfite ester compounds or their products are formulated into solutions for inhibiting water evaporation, the concentration of the sulfite ester compounds in the solution can be selected according to actual needs.

In some specific embodiments of the present invention, the application concentration of the sulfite ester compound is not less than 0.001% (w/w).

In some other specific embodiments of the present invention, the application concentration of the sulfite ester compound is 0.001% (w/w).

In some specific embodiments of the present invention, when applied, it further comprises a surfactant and/or a solvent.

In some specific embodiments of the present invention, when applied, the mass ratio of the sulfite ester compound to the surfactant is 1:0.1-20, and the concentration of the surfactant is not less than 0.001% (w/w); with the balance being solvent.

In some embodiments of the present invention, the surfactant is selected from one of ethoxylated castor oils, sodium lauryl sulfates, saponins, ethoxylated alcohols, ethoxylated fatty esters, alkoxylated diols, ethoxylated fatty acids, carboxylated alcohols, carboxylic acids, fatty acids, ethoxylated alkylphenols, fatty esters, sodium dodecyl sulfide, and Tween or a combination of more than two of them; and

The solvent is selected from one of water, ethanol, isopropanol, benzyl alcohol, acetone, acetophenone, water, glycerol, castor oils, ethoxylated alcohols, ethoxylated amides, glycerides, butanol, 1-propanol, hexanol, other alcohols, dimethyl ether, and polyethylene glycol or a combination of more than two of them.

In some specific embodiments of the present invention, the sulfite ester compound is prepared into an agricultural product, which further comprises one or more of an auxiliary dispersant, a wetting agent, a binder, an emulsifier, a stabilizer, and a solvent.

In the present invention, the dosage form includes but is not limited to missible oil, water aqua, emulsion in water, soluble agent, microemulsion, suspension concentrate, oil suspension concentrate agent, microcapsule suspension concentrate agent, and mother liquor, etc.

In some specific embodiments of the present invention, the agricultural products made from the sulfite ester compounds are combined with foliar fertilizers, water-soluble fertilizers, compound fertilizers, pesticides, plant regulators, biostimulants, and soil conditioners.

In some specific embodiments of the present invention, when in use, the sulfite compounds or the agricultural products made therefrom are used for seed treatment, foliar application or root-irrigation; preferably foliar application.

The present invention also provides a composition for inhibiting water evaporation, comprising the sulfite ester compound as described above.

In some specific embodiments of the present invention, the composition also includes agriculturally acceptable carriers or additives.

In some specific embodiments of the present invention, the carrier is water or an organic solvent; and the additives include a dispersant, a wetting agent, a binder, an emulsifier, and a stabilizer.

The present invention also provides an anti-evaporation agent, and the active ingredients of which comprise sulfite ester compounds, and the structural formula of the sulfite ester compounds is as follows:

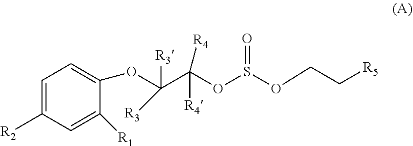

Wherein, R₁, and R₂ are selected from the group consisting of halogen;
R₃, R₃′, R₄, and R₄′ are respectively independently selected from the group consisting of hydrogen, C₁-C₅ alkyl, and C₁-C₅ alkenyl; and
R₅ is selected from a group consisting of halogen.

In some specific embodiments of the present invention, the R₁, and R₂ are selected from the group consisting of Cl; and R₅ is selected from the group consisting of F.

In some specific embodiments of the present invention, R₃, and R₃′ are selected from the group consisting of hydrogen; and R₄, and R₄′ are respectively independently selected from the group consisting of hydrogen, —CH₃, —CH₂CH₃, and —CH═CH₂.

In some specific embodiments of the present invention, the sulfite ester compound is selected from one of the following compounds or a combination of more than two of them:

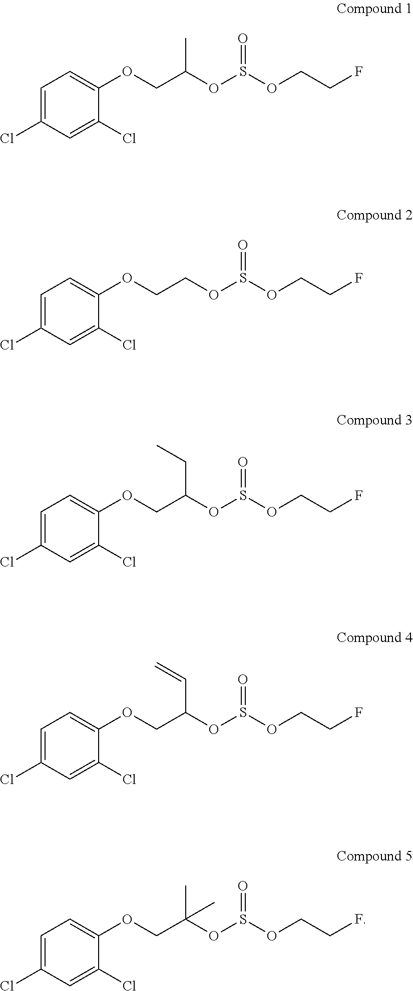 [SMILES: CC(COc1ccc(Cl)cc1Cl)OS(=O)OCCF ; O=S(OCCF)OCCOc1ccc(Cl)cc1Cl ; CCC(COc1ccc(Cl)cc1Cl)OS(=O)OCCF ; C=CC(COc1ccc(Cl)cc1Cl)OS(=O)OCCF ; CC(C)(COc1ccc(Cl)cc1Cl)OS(=O)OCCF]

In some specific embodiments of the present invention, the sulfite ester compound is selected from one of the following compounds or a combination of more than two of them:

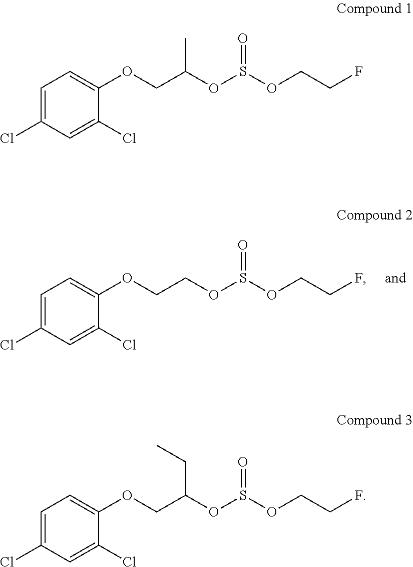 [SMILES: CC(COc1ccc(Cl)cc1Cl)OS(=O)OCCF ; O=S(OCCF)OCCOc1ccc(Cl)cc1Cl ; CCC(COc1ccc(Cl)cc1Cl)OS(=O)OCCF]

### Beneficial Effects:

(1) Highly efficient performance in inhibiting water evaporation: within 30 minutes at 30° C., the droplet evaporation rate decreased from 48.70% in the blank group to 24.06%, with an inhibition efficiency exceeding 50%; under conditions of high temperature (40° C.), the stable inhibitory effect was maintained (the evaporation rate decreased from 67.78% to 33.02%).

(2) Environmentally friendly and synergistic effects: the composition contains no heavy metals or degradation-resistant components, and there is no risk of phytotoxicity to plants; after compounding with a surfactant, the droplet spreading area increases by 25%-30%, and the pesticide utilization rate increases by 10%-15%, achieving both water-saving and precision pesticide application effects.

(3) Economical applicability and flexible operation: the synthesis process is simple (three-step reaction, with a yield ≥70%), and the raw material is of low cost. It is suitable for multiple application methods such as foliar spraying and trickle irrigation, etc., and is suitable for complex agricultural environments such as drought and high temperatures.

### DESCRIPTION OF DRAWINGS

FIG. 1 shows the inhibitory effects of different compounds on droplet evaporation (pendant-drop method);

FIG. 2 shows the inhibitory effects of different compounds on droplet evaporation (sessile drop method);

FIG. 3 shows the inhibitory effects of compound 1 at different concentrations on droplet evaporation; and

FIG. 4 shows the fitted curve graph.

### DETAILED DESCRIPTION

The technical solutions in the embodiments of the present invention will be described clearly and completely below. Obviously, the described embodiments are only part of the embodiments of the present invention rather than all of them. Based on the embodiments in the present invention, all other embodiments obtained by those ordinarily skilled in the art without creative work belong to the scope of protection of the present invention.

In the present invention, the term “inhibiting water evaporation” refers to the reduction in water loss caused by evaporation of water.

In the present invention, the “C₁-C₅ alkyl” refers to a straight or branched saturated hydrocarbon alkyl group, including at least —CH₃, —CH₂CH₃, —CH₂CH₂CH₃, —CH₂(CH₃)₂, —CH₂CH₂CH₂CH₃, —C(CH₃)₃, —CH₂C(CH₃)₃, —CH₂CH₂CH₂CH₂CH₃, etc.

In the present invention, the “C₁-C₅ alkenyl”, includes at least —CH═CH₂, —CH₂—CH═CH₂, —CH₂CH₂—CH═CH₂, and —CH₂CH₂CH₂—CH═CH₂, etc.

In the present invention, the “halogen” is selected from the group consisting of F, Cl, Br, and I.

The intermediate compound of the present invention can be prepared through various synthesis methods well-known to those skilled in the art, including the specific embodiments listed herein, the embodiments formed by combining the specific embodiments with other chemical synthesis methods, and equivalent substitutions well-known to those skilled in the art, and the preferred embodiments include but are not limited to the embodiments of the present invention.

The chemical reactions of the specific embodiments of the present invention are carried out in suitable solvents, which must be suitable for the chemical changes of the present invention and the required reagents and materials. In order to obtain the compounds of the present invention, sometimes it is necessary for those skilled in the art to modify or select the synthesis steps or reaction procedures based on the existing embodiments.

In the present invention, the application concentration of the sulfite ester compounds is not less than 0.001% (w/w), for example it can be selected from the group consisting of: 0.001%, 0.004%, 0.005%, 0.01%, 0.05%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, and 80%, etc.

In some embodiments of the present invention, the application concentration of the sulfite ester compounds can be selected from the group consisting of 0.001%-50% (w/w).

In some other embodiments of the present invention, the application concentration of the sulfite ester compounds can be selected from the group consisting of 0.001%-10% (w/w).

In some other embodiments of the present invention, the application concentration of the sulfite ester compounds can be selected from the group consisting of 0.001%-5% (w/w).

In some other embodiments of the present invention, the application concentration of the sulfite ester compounds can be selected from the group consisting of 0.001%-1% (w/w).

In some other embodiments of the present invention, the application concentration of the sulfite ester compounds can be selected from the group consisting of 0.001%-0.5% (w/w).

In some other embodiments of the present invention, the application concentration of the sulfite ester compounds can be selected from the group consisting of 0.001%-0.1% (w/w).

In some other embodiments of the present invention, the application concentration of the sulfite ester compounds can be selected from the group consisting of 0.001%-0.05% (w/w).

In the present invention, “w/w” represents weight by weight, i.e., the percentage of solute mass to the total mass of solution. For example, 0.05% (w/w) refers to a solution containing 0.05 grams of the target compound per 100 grams, with the remainder being solvents or additives.

Suitable surfactants can be selected by those skilled in the art according to actual usage requirements. Examples of surfactants that can be used in some embodiments of the present invention include but are not limited to ethoxylated castor oils, sodium lauryl sulfates, saponins, ethoxylated alcohols, ethoxylated fatty esters, alkoxylated diols, ethoxylated fatty acids, carboxylated alcohols, carboxylic acids, fatty acids, ethoxylated alkylphenols, fatty esters, sodium dodecyl sulfide, Tween, other surfactants based on fatty acids, other natural or synthetic surfactants, and combinations thereof. In some embodiments, the surfactant is a non-ionic surfactant. In some embodiments, the surfactant is an ionic surfactant. The selection of appropriate surfactants depends on the relevant applications and usage conditions, and appropriate surfactants are known to those skilled in the art.

In some embodiments of the present invention, the surfactant is selected from the group consisting of Tween, and the commonly used Tween models include but are not limited to Tween-20, Tween-60, and Tween-80.

In some embodiments of the present invention, when applied, the mass ratio of sulfite ester compound to surfactant is 1:0.1-20, for example, 1:0.1, 1:0.5, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20.

In some embodiments of the present invention, when applied, the concentration of the surfactants is not less than 0.001% (w/w), for example it can be selected from the group consisting of: 0.001%, 0.004%, 0.005%, 0.01%, 0.05%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, and 50%, etc.

In some embodiments of the present invention, when applied, the application concentration of the surfactant can be selected from the group consisting of 0.001%-50% (w/w).

In some embodiments of the present invention, when applied, the application concentration of the surfactant can be selected from the group consisting of 0.001%-10% (w/w).

In some other embodiments of the present invention, when applied, the application concentration of the surfactant can be selected from the group consisting of 0.001%-5% (w/w).

In some other embodiments of the present invention, when applied, the application concentration of the surfactant can be selected from the group consisting of 0.001%-10% (w/w).

In some other embodiments of the present invention, when applied, the application concentration of the surfactant can be selected from the group consisting of 0.001%-0.5% (w/w).

In some other embodiments of the present invention, when applied, the application concentration of the surfactant can be selected from the group consisting of 0.0010%-0.10% (w/w).

In some embodiments of the present invention, when applied, the proportion of solvent is greater than 1% (w/w), for example it can be selected from the group consisting of: 1%, 2%, 3%, 4%, 5%, 6%, 7%, 8%, 9%, 10, 1%, 12%, 13%, 14%, 15%, 16%, 17%, 18, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 99%, 99.9%, and 99.99%, etc.

In some other embodiments of the present invention, when applied, the proportion of solvent can be 50-99.99% (w/w).

A dispersant is a chemical additive that promotes the uniform dispersion of solid particles in liquids or matrices by reducing the interaction force among particles. In agricultural preparations, the dispersant is commonly used for suspension concentrates (SCs) and granules (GRs). For example, in aqueous suspension concentrates, the dispersant such as sodium lignosulphonate or polycarboxylates can stably disperse ultrafine particles (with a particle size of <4 microns) of solid raw pesticides in water, avoiding particle aggregation or sinking, thereby ensuring uniform distribution of the physic liquor during spraying. In granules, the dispersant helps to evenly mix the raw pesticides with the carrier (such as clay), preventing stratification of components during granulation, and ensuring the consistency of active ingredients inside the particles.

A wetting agent is a type of surfactant that enhances the wetting and permeation ability of physic liquor on solid surfaces by reducing the surface tension of the liquid. Its core role is to promote the rapid dispersion of hydrophobic powders or particles in water, and to improve the spreading uniformity of the physic liquor on the foliar surface, avoiding droplet rebound.

For example, in wettable powders (WPs), sodium dodecyl sulfate (SDS) rapidly wets imidacloprid powder to form a suspension concentrate; when it is applied by foliar spraying, the natural wetting agent saponin can improve the adhesion effect of the physic liquor on waxy leaves and reduce loss.

A binder is an additive that enhances the mechanical strength of particles or powders through physical or chemical action, preventing crushing or dust generation. In granules (GRs), the binders such as polyvinyl alcohol, sodium carboxymethyl cellulose, or starch bond the raw pesticides with a carrier such as diatomaceous earth to form a robust granular structure. For example, adding polyvinyl alcohol as a binder to herbicide granules can significantly increase particle hardness, reduce pulverization rate during transportation, meanwhile ensuring slow disintegration of particles in the field, thereby prolonging the persistence of pesticide efficacy.

An emulsifier is a kind of surfactant that can promote two-phase mixing and form stable emulsion by reducing the interfacial tension between oil and water. In emulsions (ECs), the emulsifier such as Tween-80, AEO-9, or alkylphenol ethoxylate dissolves high concentration of the raw pesticides in an organic solvent such as xylene and mixes with water to form a homogeneous emulsion. For example, the addition of Tween-80 as an emulsifier in Chlorpyrifos emulsifiable concentrate can form a stable emulsion after dilution to avoid oil-water stratification and ensure that the physic liquor evenly covers the surface of crops. In addition, the emulsifier can also be used for microemulsions (MEs), and the nanoscale emulsion dispersion can be realized by reducing the interfacial tension.

A stabilizer is a type of functional additive used to maintain the chemical stability such as anti-decomposition and physical stability such as anti-sedimentation and anti-stratification of preparations. In suspension concentrates (SCs), stabilizers such as xanthan gum, magnesium aluminum silicate, or bentonite can prevent solid particles from settling or agglomeration, extending the shelf life. For example, adding xanthan gum to a bactericidal suspension concentrate can significantly improve the stability of the suspension and avoid stratification during storage. In granules, stabilizers such as antioxidant BHT or light stabilizer UV-326 can prevent the raw pesticides from oxidation or photolysis in the granules, ensuring long-lasting pesticide effect. For example, insecticidal granules containing BHT can still maintain the activity of active ingredients in high temperature environments.

Missible oil is a type of agricultural preparation, which is a liquid made by dissolving high concentrations of active ingredients in a solvent and adding an emulsifier. Generally, after the missible oil is diluted with a large amount of water to form a stable emulsion, it can be dispersed with a sprayer, or it can be sprayed in a low volume or even ultra-low volume. It can be used directly or diluted with water before spraying.

Water aqua is a solution of raw pesticides, and the agent is uniformly dispersed in water in the state of ions or molecules. The concentration of the agent depends on the solubility of the raw pesticides in water, which is generally its maximum solubility, and it is diluted with water when used.

Emulsion in water is a liquid raw pesticide made by mixing liquids or solvents, uniformly dispersing in water as small droplets of 0.5-1.5 microns, with a milky white appearance. It is composed of active ingredients, emulsifiers, dispersants, and antifreeze agents, etc. Its active ingredients usually require a water solubility of less than 1000 mg/L, and the emulsifier can reduce surface and interfacial tension, so that the oil phase is dispersed into tiny oil droplets, which can stably exist in the water phase, and the coordination of a dispersant with a thickener can improve the low-temperature and freeze-thaw stability, and the antifreeze agent can improve the low-temperature stability of the preparation.

A soluble agent is a pesticide dosage form. It is prepared by dissolving an active ingredient in a suitable solvent to form a homogeneous, transparent liquid dosage form. The active ingredients in such a dosage form usually have good water solubility or high solubility in specific solvents.

Microemulsion is a thermodynamically stable, isotropic, transparent or semi-transparent liquid dosage form. It is a colloidal dispersion system with a particle size between 0.01 and 0.1 microns formed by mixing components such as raw pesticides, surfactants, cosurfactants, and water, etc., under appropriate conditions. The microemulsion has high stability and is not prone to phenomena such as stratification, stratification, and the like during storage. Its appearance is transparent or semitransparent, similar to microemulsions, which enables it to be more evenly dispersed in water when used, forming a stable emulsion, which is conducive to accurate measurement and dilution.

A suspension concentrate refers to a preparation in which solid raw pesticides are uniformly dispersed in water in particulates of less than 4 microns, with the international code SC, which has fine particle size, generally ranging from 0.1-3 m, and the suspension rate is high. The suspension concentrates are divided into two types: aqueous suspension concentrates and oily suspension concentrates. Aqueous suspension concentrate uses water as the suspension medium, while oil suspension concentrate uses oil as the suspension medium and does not contain water. The commonly used oils are vegetable oils, such as corn oil, and rapeseed oil, etc. The suspension concentrate can completely eliminate the use of organic solvents and is a good dosage form for processing solid raw pesticides. The suspension concentrate is a mixture of solid powder and liquid suspended in water, which needs to be shaken well before use, then diluted with water, and sprayed. The suspension concentrate is easy to carry and dilute, and can be evenly sprayed, which has good adhesion and persistence.

Oily suspension concentrate is a dosage form in which insoluble raw pesticides are uniformly dispersed in a tiny particle state in an oil phase medium. It is mainly composed of raw pesticides, oil carriers, dispersants, emulsifiers, and thickeners, etc. Vegetable oils or mineral oils are usually selected as oily carriers, which provide a dispersion medium for raw pesticides. The function of dispersant and emulsifier is to evenly disperse and stabilize the particles of raw pesticides in the oil phase, preventing particle aggregation and settling. While thickener is used to adjust the viscosity of the preparation, making it have good fluidity and stability.

Microcapsule suspension concentrate is a dosage form that encapsulates the active ingredients in tiny capsule walls, forming microcapsules with a certain particle size, and suspending them in the aqueous phase. It is composed of various components such as raw pesticides, capsule wall materials, emulsifiers, dispersants, solvents, and water, etc.

Microcapsule suspension concentrate has many advantages. Firstly, it can effectively prolong the duration of pesticide efficacy, allowing the pesticide to continue to exert its efficacy for a longer period of time through the slow release of the capsule wall. Secondly, it can reduce the toxicity and irritation of pesticides. As the active ingredients are encapsulated in the capsule, direct contact with the external environment and organisms is reduced, resulting in higher safety for humans, livestocks and beneficial organisms.

Mother liquor is a concentrated system formed by pre-mixing effective ingredients with solvents, and additives, etc., in specific proportions. Its appearance can be a transparent solution, suspension, or viscous paste depending on the composition. For example, missible oil mother liquor is often composed of raw pesticides (such as pyrethroids), solvents (xylene) and emulsifiers (such as alkylphenol ethoxylate), when in use, it is diluted to spray solution by adding water in proportion.

In the present invention, the sulfite ester compounds can be used in combination with foliar fertilizers, water-soluble fertilizers, compound fertilizers, pesticides, plant regulators, biostimulants, and soil conditioners, and are adaptable to the application methods such as foliar spraying, seed treatment, and root-irrigation, etc., to achieve comprehensive efficiency enhancement in agricultural production. (1) collaborative mechanism of combination: when compounded with foliar fertilizers or pesticides, the compounds can prolong the retention time of droplets by inhibiting evaporation and improve nutrient or absorption efficiency of active ingredients. For example, spraying tomato with humic acid foliar fertilizer can enhance fruit quality; when combined with water-soluble fertilizer for trickle irrigation, it forms a water retention barrier on the surface of soil, optimizing the slow-release performance of nitrogen fertilizer, and reducing irrigation demand; when combined with pesticides, it improves the prevention and control effect in high-temperature environments by delaying droplet drying, while reducing the frequency of application and residual risk; (2) multi-route application effects: foliar spraying (preferred): a uniform molecular film will be formed, reducing water evaporation and transpiration intimidation, such as protecting crops from burns in high-temperature and arid regions; seed treatment: the film-forming properties promote germination and root development, such as significantly enhancing the drought resistance and soil borne disease resistance of wheat after seed soaking; root irrigation application: a soil water retaining layer is constructed, extending the watering and fertilizing utility cycle, such as promoting vigorously root growth in corn field applications.

The “application” as described in the present invention refers to the technical behaviors of adding specific substances (such as fertilizers, pesticides, and amendments, etc.) to soil, plants, or the environment through manual or mechanical means, in order to improve crop growth conditions, prevent and control pests and diseases, regulate soil properties, or optimize agricultural production, etc.

The “foliar fertilizer” as described in the present invention refers to a type of fertilizer that can be directly absorbed by leaves and participate in plant metabolism by applying to the surface of plant leaves by spraying. It is characterized by bypassing the soil and directly acting on the plant body, which can quickly supplement nutrients, especially when root absorption is limited, or nutrient deficiency symptoms need to be corrected in a timely manner. For example, water-soluble solutions containing amino acids and trace elements belong to this category.

The “water-soluble fertilizer” as described in the present invention is a multi-element compound fertilizer that is completely soluble in water. It usually comprises macroelements such as nitrogen, phosphorus, and potassium, and medium trace elements such as calcium, magnesium, and iron, etc., partially added with organic components such as amino acids and humic acids, etc. It is characterized by being suitable for integrated water and fertilizer technologies such as sprinkling irrigation and trickle irrigation, etc., with quick action, formula flexibility, and high utilization rate.

The “compound fertilizer” as described in the present invention is a type of compound fertilizer that contains at least two of the three nutrients: nitrogen, phosphorus, and potassium. It has a fixed nutrient ratio, and is suitable for soil-based fertilization, and complementary to foliar fertilizer or water-soluble fertilizer. For example, potassium dihydrogen phosphate containing phosphorus and potassium belongs to this category.

The “pesticide” as described in the present invention is a chemical or biological agent used for preventing, eliminating, or controlling agricultural and forestry pests and diseases, as well as regulating plant growth. Categories such as insecticides, bactericides, herbicides, and plant growth regulators, etc., are included. It is necessary to pay attention to the difference from foliar fertilizers that pesticides are mainly used to prevent and control harmful organisms, while foliar fertilizers are mainly used to supplement nutrients, however both of them can be mixed to improve efficiency.

The “plant growth regulator” as described in the present invention is a type of active substances that affect the development of plants by regulating their physiological processes such as growth, flowering, and fruiting. It includes natural hormones such as auxin, gibberellin and artificially synthesized analogues, etc., they need to be distinguished from fertilizers because their functions focus on physiological regulation rather than nutrient supply. For example, chitosan, as a biostimulant, can enhance stress resistance of plants.

The “biostimulant” as described in the present invention is a substance or microorganism that improves plant growth by promoting nutrient absorption, enhancing resistance to abiotic stress such as drought and salinization, or improving quality.

The “soil conditioner” as described in the present invention is a material used to improve the physical, chemical, or biological properties of soil, such as adjusting pH, increasing organic matters, or promoting beneficial microbial activity. Its core function is to optimize the soil environment rather than directly providing nutrients.

The term “including” or “comprising” as used in the present application shall be interpreted in its open-ended, meaning that it is specified that there are specified features, elements, steps, or components as mentioned, but it does not exclude the presence or addition of further features, elements, steps, or components.

In the present invention, the “solvent” used can be selected from water, ketones, alcohols, aldehydes, ethers, esters, or carboxylic acids, and can include non-aryl ketones, non-aryl alcohols, non-aryl aldehydes, non-aryl esters, non-aryl carboxylic acids, aryl alcohols, aryl-alkyl alcohols, aryl aldehydes, aryl-alkyl ketones, aryl-aryl ketones, aryl carboxylic acids, aryl-alkyl esters, aryl-aryl ethers, aryl-alkyl ethers, aryl-aryl ethers, and/or combinations thereof.

In some embodiments, the “organic solvent” can be selected from hydrocarbons, halohydrocarbons, oxygen-containing solvents, natural source solvents, special functional solvents, and their compounded systems, such as n-hexane, toluene, dichloromethane, ethanol, acetone, ethyl acetate, castor oil, and turpentine, etc.

In some embodiments, the solvent includes water, ethanol, isopropanol, benzyl alcohol, acetone, acetophenone, water, glycerol, castor oil, ethoxylated alcohols, ethoxylated amides, glycerides, butanol, 1-propanol, hexanol, other alcohols, dimethyl ether, polyethylene glycol, etc.

### Embodiment 1: Synthesis of Sulfite Ester Compounds

### (1) Synthesis of Compound 1

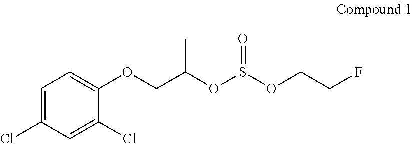 [SMILES: CC(COc1ccc(Cl)cc1Cl)OS(=O)OCCF]

### Step 1:

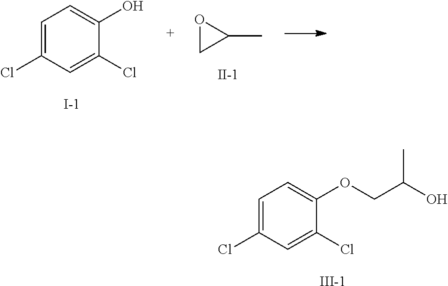

2,4-dichlorophenol (1 g, 6.2 mmol) was added into a reaction flask, and dissolved in 20 mL of DMF, then propylene oxide (722 mg, 7.6 mmol), and cesium carbonate (8 g, 24.8 mmol) were added therein, and heated and reacted in an oil-bath pan at 100° C. After 6 hours, the reaction was monitored by TLC to be completed, then 100 mL of water was added and extracted with ethyl acetate (30 mL×3), washed with saturated saline solution and the organic phase was collected, dried over anhydrous sodium sulfate, and concentrated under reduced pressure, then compound iii-1 (989 mg) as colorless transparent liquid was obtained by separation and purification.

### Step 2:

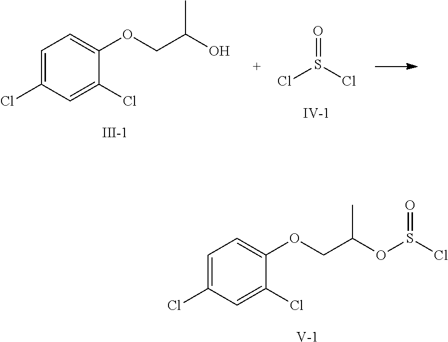

Thionyl chloride (803 mg, 6.8 mmol) was added into a reaction flask, and dissolved in 20 mL of dichloromethane, the reaction flask was transferred to an ice bath at 0° C. and stirred, then compounds iii-1 (989 mg, 4.5 mmol) was added dropwise slowly therein while stirring. After the dropwise addition was completed, the reaction flask was warmed to room temperature and reacted for 10 hours. The reaction was monitored by TLC until it was completed, and the reaction solution was concentrated under reduced pressure to obtain a light-yellow oil, which was the crude product of compound v-1, and was ready for later use.

### Step 3:

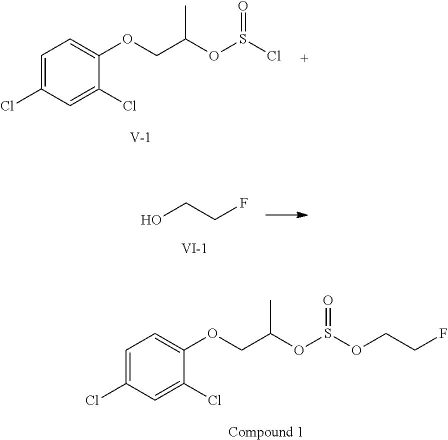

Compound vi-1 (346 mg, 5.4 mmol) was added into a reaction flask, and triethylamine (683 mg, 6.8 mmol) was added, the reaction flask was transferred to an ice bath at 0° C. and stirred, then compounds v-1 (1269 mg, 4.5 mmol) was added dropwise slowly therein. After the dropwise addition was completed, the reaction flask was warmed to room temperature and reacted for 6 hours, then the reaction was monitored by TLC until it was completed, then 100 mL of water was added to the reaction solution and extracted with dichloromethane (30 mL×3), which was washed with saturated saline solution, then the organic phase was collected, dried over anhydrous sodium sulfate, and concentrated under reduced pressure, then compound 1 (902 mg) as homogeneous liquid was obtained by purifying via column chromatography.

¹H NMR (400 MHz, CDCl3) δ 7.39 (d, J=2.5 Hz, 1H), 7.20 (dd, J=8.8, 2.5 Hz, 1H), 6.85 (d, J=8.8 Hz, 1H), 5.01-4.93 (m, 1H), 4.74-4.66 (m, 1H), 4.63-4.55 (m, 1H), 4.40-4.31 (m, 1H), 4.31-4.22 (m, 1H), 4.10 (dd, J=10.0, 6.3 Hz, 1H), 4.02 (dd, J=10.0, 4.3 Hz, 1H), 1.52 (d, J=6.5 Hz, 3H) ppm.

HRMS (ESI) Calcd. For CllHi₄Cl₂FO₄SNa+[M+Na]+352.9768; Found: 352.9786, 354.9758.

### (2) Synthesis of Compound 2

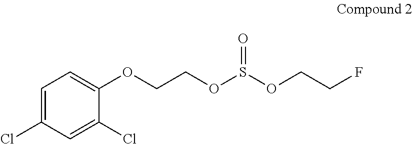 [SMILES: O=S(OCCF)OCCOc1ccc(Cl)cc1Cl]

By referring to the synthesis method of compound 1, wherein the propylene oxide in step 1 was replaced with ethylene carbonate, and compound 2 was synthesized through steps 2 and 3.

¹H NMR (400 MHz, CDCl₃) δ 7.31 (d, J=2.5 Hz, 1H), 7.12 (dd, J=8.8, 2.5 Hz, 1H), 6.78 (d, J=8.8 Hz, 1H), 4.65-4.62 (m, 1H), 4.53-4.49 (m, 1H), 4.34-4.30 (m, 2H), 4.25-4.18 (m, 3H) ppm.

HRMS (ESI) Calcd. For C₁₀H₁₁O₄Cl₂FS⁺[M+H]⁺ 316.9739; Found:316.09658.

### (3) Synthesis of Compound 3

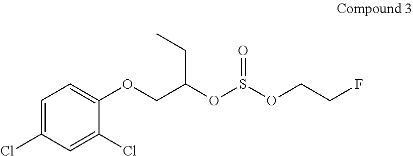 [SMILES: CCC(COc1ccc(Cl)cc1Cl)OS(=O)OCCF]

By referring to the synthesis method of compound 1, wherein the propylene oxide in step 1 was replaced with 1,2-butylene oxide, and compound 3 was synthesized through steps 2 and 3.

¹H NMR (400 MHz, CDCl₃) δ 7.32-7.29 (m, 1H), 7.14-7.09 (m, 1H), 6.85-6.81 (m, 1H), 4.75-3.96 (m, 7H), 1.86-1.66 (m, 2H), 0.98 (t, J=6.7 Hz, 3H) ppm.

HRMS (ESI) Calcd. For C₁₂H₁₅O₄Cl₂FS⁺[M+H]⁺ 344.0052; Found:344.0026.

### (4) Synthesis of Compound 4

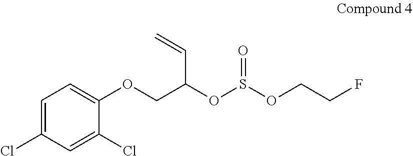 [SMILES: C=CC(COc1ccc(Cl)cc1Cl)OS(=O)OCCF]

By referring to the synthesis method of compound 1, wherein the propylene oxide in step 1 was replaced with butadiene monoxide, and compound 4 was synthesized through steps 2 and 3.

¹H NMR (400 MHz, CDCl₃) δ 7.31 (dd, J=2.6, 1.4 Hz, 1H), 7.11 (dd, J=8.8, 2.5 Hz, 1H), 6.77 (dd, J=8.8, 1.2 Hz, 1H), 5.95-5.87 (m, 1H), 5.48 (d, J=17.2, Hz, 1H), 5.39-5.33 (m, 1H), 5.17-5.10 (m, 1H), 4.65-4.56 (m, 1H), 4.54-4.45 (m, 1H), 4.40-4.12 (m, 2H), 4.06-3.97 (m, 2H) ppm.

### (5) Synthesis of Compound 5

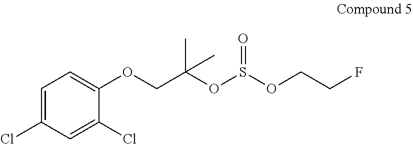 [SMILES: CC(C)(COc1ccc(Cl)cc1Cl)OS(=O)OCCF]

By referring to the synthesis method of compound 1, wherein the propylene oxide in step 1 was replaced with methylpropylene oxide, and compound 5 was synthesized through steps 2 and 3.

¹H NMR (400 MHz, CDCl₃) δ 7.31 (d, J=2.5 Hz, 1H), 7.11 (dd, J=8.8, 2.6 Hz, 1H), 6.75 (d, J=8.8 Hz, 1H), 4.61-4.57 (m, 1H), 4.49-4.45 (m, 1H), 4.30-2.34 (m, 1H), 4.21-4.18 (m, 1H), 3.92 (s, 2H), 1.58 (s, 3H), 1.57 (s, 3H) ppm.

HRMS (ESI) Calcd. For C₁₂H₁₆Cl₂FO₄S⁺[M+H]⁺ 345.0125; Found:345.0143.

### Test Example 1: Pendent Drop Method for Testing Droplet Evaporation Inhibition Rate

### 1. Sample Preparation:

CK (blank group): 50% (w/w) Tween 80, 50% (w/w) deionized water, an equal amount of deionized water and Tween 80 were mixed to form a homogeneous liquid, and the solution to be tested of blank group was prepared for later use;
Methylated soybean oil: 50% (w/w) Tween 80, 50% (w/w) methylated soybean oil, an equal amount of methylated soybean oil and Tween 80 were mixed to form a homogeneous liquid, and the solution to be tested was prepared for later use;
Compound 1: 50% (w/w) tween 80, 50% (w/w) compound 1, an equal amount of compound 1 and Tween 80 were mixed to form a homogeneous liquid, and the solution to be tested was prepared for later use;
Compound 2: 50% (w/w) tween 80, 50% (w/w) compound 2, an equal amount of compound 2 and Tween 80 were mixed to form a homogeneous liquid, and the solution to be tested was prepared for later use;
Compound 3: 50% (w/w) tween 80, 50% (w/w) compound 3, an equal amount of compound 3 and Tween 80 were mixed to form a homogeneous liquid, and the solution to be tested was prepared for later use;
Compound 4: 50% (w/w) tween 80, 50% (w/w) compound 4, an equal amount of compound 4 and Tween 80 were mixed to form a homogeneous liquid, and the solution to be tested was prepared for later use;
Compound 5: 50% (w/w) tween 80, 50% (w/w) compound 5, an equal amount of compound 5 and Tween 80 were mixed to form a homogeneous liquid, and the solution to be tested was prepared for later use.

### 2. Experimental Method

(1) 0.1 g+0.001 g of the solution to be tested was weighed and 1000-fold diluted with standard deionized water to 100 g to form the sample to be tested;
(2) After the sample to be tested was aspirated with a microsyringe, the microsyringe was fixed in the syringe holder slot of the contact angle meter, and vertically fixed above the temperature control box, and the needle of the microsyringe was inserted into the interior of the temperature control box to avoid the influence of environmental temperature and humidity on droplet evaporation, with the temperature of the water bath connected to the temperature control box being 30° C.;
(3) After the instrument reached a stable state, a single droplet of 15 μL was generated through the droplet generator on the measuring instrument that was controlled by SCA20 software, at which point the droplet was suspended on the needle of the microsyringe;
(4) Video was selected and recorded, and the droplet was automatically shot through the CCD lens of the image acquisition device, and the entire process of droplet evaporation was recorded; by using SCA20 software for data processing, the change in droplet volume over time was obtained, and the droplet evaporation rate was calculated using a formula.

The data of droplet evaporation rate and droplet evaporation inhibition rate in this experiment were studied with an evaporation time of 30 minutes. The temperature during measurement was 30° C.

Droplet evaporation rate=(initial volume of droplet-final volume of droplet)/initial volume of droplet; Evaporation inhibition rate (%)=(W_{f}−W_{d})/W_{f}×100% (W_{f}: water evaporation amount of the blank group; W_{d}: water evaporation amount of the test group).

### 3. Analysis of Results

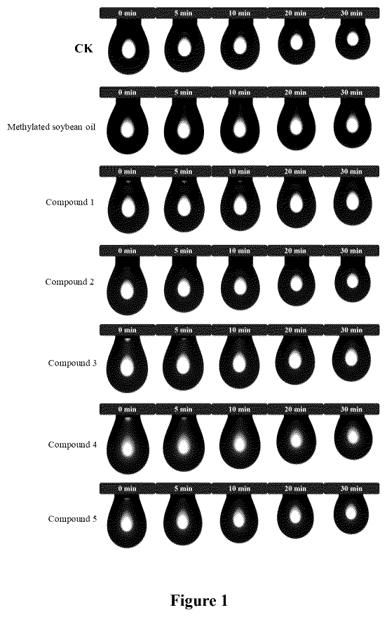

The results showed (Table 1 and FIG. 1) that the addition of compounds 1 and 3 significantly inhibited droplet evaporation. At 30 minutes, the evaporation rate of the blank group reached 48.70%, while the compounds 1 and 3 groups decreased to 24.06% and 27.79%, respectively, and both showed a linear inhibitory trend (R²>0.98) within 30 minutes, and compounds 2 (37.33%), 4 (38.26%), and 5 (43.00%) also exhibited certain inhibitory effects; in addition, compound 1 had the highest evaporation inhibition rate (50.60%), which was significantly better than methylated soybean oil (49.92%) and other compounds; compound 3 had the second highest inhibition rate of 42.94%, which was better than compound 2 (23.36%), compound 4 (21.45%), and compound 5 (11.70%); its mechanism may involve the formation of dynamic interfacial film by intermolecular interaction, thus reducing the mass transfer efficiency at the liquid-gas interface. The inhibition rate of compound 1 is similar to that of methylated soybean oil, but compound 1 has more advantages in long-term applications due to its more stable molecular structure.

TABLE 1
Evaporation Rate and Evaporation Inhibition Rate (%)
of Different Compounds at 30 °C within 0-30 Minutes

| | | | | | | Evaporation |
|---|---|---|---|---|---|---|
| | Evaporation Rate (Reserve two | | | | | Inhibition |
| | decimal fractions) | | | | | Rate |
| | 0 min | 5 min | 10 min | 20 min | 30 min | 30 min |
| CK | 0% | 9.71% | 15.86% | 32.47% | 48.70% | / |
| Methylated | 0% | 6.03% | 13.03% | 20.65% | 24.39% | 49.92% |
| soybean oil | | | | | | |
| Compound 1 | 0% | 6.47% | 10.36% | 17.25% | 24.06% | 50.60% |
| Compound 2 | 0% | 6.46% | 11.07% | 22.36% | 37.33% | 23.36% |
| Compound 3 | 0% | 6.88% | 10.62% | 20.51% | 27.79% | 42.94% |
| Compound 4 | 0% | 8.08% | 15.09% | 25.55% | 38.26% | 21.45% |
| Compound 5 | 0% | 7.70% | 13.83% | 29.01% | 43.00% | 11.70% |

### Test Example 2: Sessile Drop Method for Testing Droplet Evaporation Inhibition Rate

### 1. Sample Preparation:

The preparation method of samples was the same as in Test Example 1.

### 2. Experimental Method

(1) 0.1 g+0.001 g of the solution to be tested was weighed and 1000-fold diluted with standard deionized water to 100 g to form the sample to be tested;

(2) In order to better simulate the evaporation of droplets on the surface of the leaves, a standard paraffin sheet was used to simulate the wax layer on the surface of the leaves. After the solution to be tested was aspirated with a microsyringe, the microsyringe was fixed in the syringe holder slot of the contact angle meter. After the instrument reached a stable state, a single droplet of 10 μL was generated through the droplet generator on the measuring instrument that was controlled by SCA20 software, the standard paraffin film was placed in a temperature control box to avoid the influence of environmental temperature and humidity on droplet evaporation, with the temperature of the water bath connected to the temperature control box being 30° C.;

(3) Video was selected and recorded, and the droplet was automatically shot through the CCD lens of the image acquisition device (with the shooting interval being 2 seconds), and the entire process of droplet evaporation was recorded; by using SCA20 software for data processing, the change in droplet volume over time was obtained, and the droplet evaporation rate was calculated using a formula.

The data of droplet evaporation rate and droplet evaporation inhibition rate in this experiment were studied with an evaporation time of 5 minutes. The temperature during measurement was 30° C.

Droplet evaporation rate=(initial volume of droplet-final volume of droplet)/initial volume of droplet.

Evaporation inhibition rate (%)=(W_{f}—W_{d})/W_{f}×100% (W_{f}: water evaporation amount of the blank group; W_{d}: water evaporation amount of the test group).

### 3 Analysis of Results

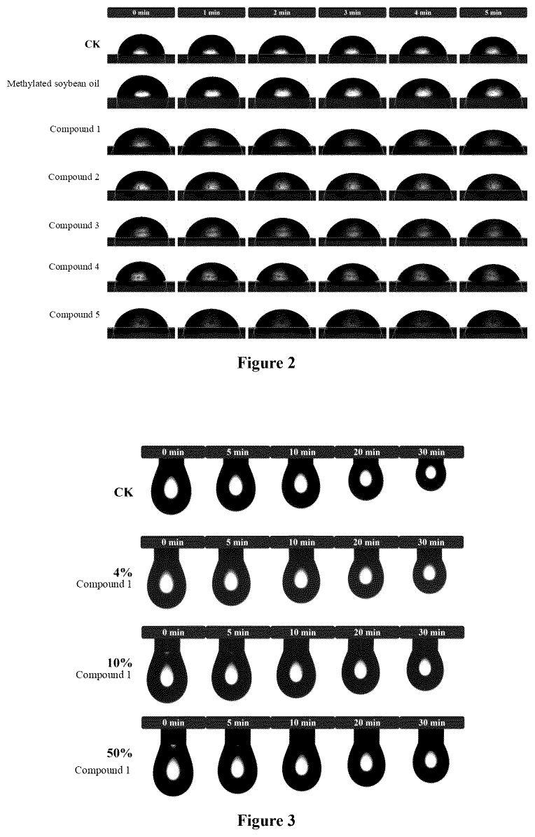

The experimental results (FIG. 2 and Table 2) showed that compound 1 and compound 2 exhibited significant evaporation inhibition effects within 5 minutes. The evaporation rate of the control group reached 16.09% within 5 minutes, while the compounds 1 and 2 groups decreased to 9.11% and 10.61%, respectively. Kinetic analysis showed that the inhibition rate of the two within 5 minutes was 43.3% and 33.0%, respectively, which was significantly better than other tested compounds. In addition, compound 1 had the highest evaporation inhibition rate (43.35%), which was significantly better than methylated soybean oil and other compounds.

TABLE 2
Evaporation Rate and Evaporation Inhibition Rate (%)
of Different Compounds at 30 °C within 0-5 Minutes

| | | | | | | | Evaporation |
|---|---|---|---|---|---|---|---|
| | | | | | | | Inhibition |
| | Evaporation Rate (Reserve two decimal fractions) | | | | | | Rate |
| | 0 min | 1 min | 2 min | 3 min | 4 min | 5 min | 5 min |
| CK | 0% | 2.98% | 6.32% | 9.65% | 12.87% | 16.09% | / |
| Methylated | 0% | 2.87% | 5.41% | 7.30% | 9.52% | 11.65% | 27.59% |
| soybean oil | | | | | | | |
| Compound 1 | 0% | 0.82% | 2.76% | 4.91% | 7.06% | 9.11% | 43.35% |
| Compound 2 | 0% | 1.62% | 3.84% | 6.06% | 8.38% | 10.61% | 33.03% |
| Compound 3 | 0% | 2.00% | 4.09% | 6.36% | 8.64% | 11.00% | 31.63% |
| Compound 4 | 0% | 0.71% | 3.77% | 6.71% | 9.42% | 12.13% | 24.61% |
| Compound 5 | 0% | 2.45% | 4.76% | 7.46% | 9.93% | 13.45% | 16.41% |

### Test Example 3: Relationship Between Compound Concentration and Anti-Evaporation Performance

### 1. Sample Preparation:

CK (blank group): 50% (w/w) Tween 80, 50% (w/w) deionized water, deionized water and Tween 80 were mixed to form a homogeneous liquid, and the solution to be tested of blank group was prepared for later use;
4% Compound 1: 50% (w/w) tween 80, 4% (w/w) compound 1, and 46% (w/w) deionized water, 0.4 g±0.001 g of compound 1 and 5 g±0.00 Ig of tween 80 were weighed and mixed to form a homogeneous liquid, then 4.6 g±0.001 g of deionized water was further added and mixed to prepare the solution to be tested for later use;
10% Compound 1: 50% (w/w) tween 80, 10% (w/w) compound 1, and 40% (w/w) deionized water, 1 g±0.001 g of compound 1 and 5 g±0.00 Ig of tween 80 were weighed and mixed to form a homogeneous liquid, then 4 g±0.001 g of deionized water was further added and mixed to prepare the solution to be tested for later use;
50% Compound 1: 50% (w/w) tween 80, 50% (w/w) compound 1, an equal amount of compound 1 and Tween 80 were weighed and mixed to form a homogeneous liquid, and the solution to be tested was prepared for later use.

### 2. Experimental Method

(1) 0.1 g+0.001 g of the solution to be tested were weighed respectively, and 1000-fold diluted with standard deionized water to 100 g to form the sample to be tested;
(2) After the sample to be tested was aspirated with a microsyringe, the microsyringe was fixed in the syringe holder slot of the contact angle meter, and vertically fixed above the temperature control box, and the needle of the microsyringe was inserted into the interior of the temperature control box to avoid the influence of environmental temperature and humidity on droplet evaporation, with the temperature of the water bath connected to the temperature control box being 40° C.;
(3) After the instrument reached a stable state, a single droplet of 15 μL was generated through the droplet generator on the measuring instrument that was controlled by SCA20 software, at which point the droplet was suspended on the needle of the microsyringe;
(4) Video was selected and recorded, and the droplet was automatically shot through the CCD lens of the image acquisition device, and the entire process of droplet evaporation was recorded; by using SCA20 software for data processing, the change in droplet volume over time was obtained, and the droplet evaporation rate was calculated using a formula.

The data of droplet evaporation rate and droplet evaporation inhibition rate in this experiment were studied with an evaporation time of 30 minutes. The temperature during measurement was 40° C.

Droplet evaporation rate=(initial volume of droplet-final volume of droplet)/initial volume of droplet.

Evaporation inhibition rate (%)=(W_{f}—W_{d})/W_{f}×100% (W_{f}: water evaporation amount of the blank group; W_{d}: water evaporation amount of the test group).

### 3. Analysis of Results

In this experimental exploration, it was found that the addition of compound 1 had a significant improvement effect on the anti-evaporation properties of the droplets. In the blank group, after an observation period of 30 minutes, the evaporation rate of the droplets had reached 67.78%, which means that more than two-thirds of the droplets evaporated completely within half an hour, and the water dissipation was quite serious. When compound 1 was added to the droplet system, the situation was greatly improved. At the same 30-minute node, the evaporation rate of droplets containing compound 1 was only within the interval range of 30%-50%; at 30 minutes, its evaporation inhibition rate reached a maximum of 51.30%, demonstrating its excellent anti-evaporation effect, effectively slowing down the rate of droplet water loss and enhancing the stability of droplet (FIG. 3 and Table 3)

TABLE 3
Evaporation Rate and Evaporation Inhibition Rate (%)
of Compound 1 at Different Concentrations at 40 °C

| | | | | | | Evaporation |
|---|---|---|---|---|---|---|
| | | | | | | Inhibition |
| | Evaporation Rate (Reserve two decimal fractions) | | | | | Rate |
| | 0 min | 5 min | 10 min | 20 min | 30 min | 30 min |
| CK | 0% | 8.87% | 17.98% | 42.23% | 67.78% | / |
| 4% Compound 1 | 0% | 7.57% | 18.26% | 33.13% | 46.43% | 31.53% |
| 10% Compound 1 | 0% | 6.58% | 13.48% | 25.76% | 36.20% | 46.59% |
| 50% Compound 1 | 0% | 5.84% | 13.63% | 24.84% | 33.02% | 51.30% |

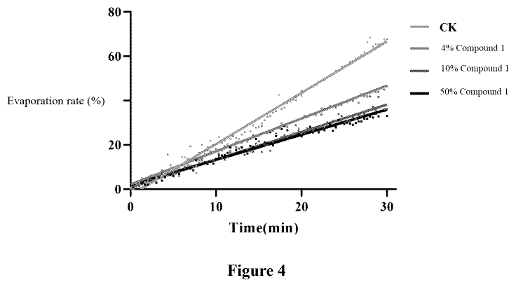

Linear fitting processing on the evaporation rate data of droplets measured at each period was further performed. The trend presented by fitting the curve, an extremely obvious pattern can be observed, i.e., as the additional amount of compound 1 gradually increases, the evaporation rate of the droplets shows a significant decreasing trend. This fully indicates that there is a close relationship between the addition amount of compound 1 and the anti-evaporation ability of the droplet. The more it is added, the stronger the anti-evaporation ability of the droplet (FIG. 4).

Although the embodiments of the present invention have been illustrated and described, it will be understood by those of ordinary skill in the art that various changes, modifications, substitutions, and variations can be made to these embodiments without departing from the principles and spirit of the present invention. The scope of the present invention is limited by the appended claims and their equivalents.

1. A method for inhibiting water evaporation, characterized in that applying sulfite ester compounds to water, the structural formula of the sulfite ester compounds is as follows: 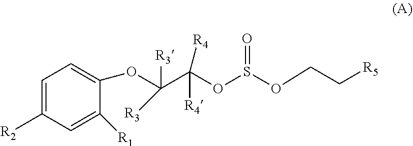
Wherein, R₁, and R₂ are selected from the group consisting of halogen;
R₃, R₃′, R₄, and R₄′ are respectively independently selected from the group consisting of hydrogen, C₁-C₅ alkyl, and C₁-C₅ alkenyl; and
R₅ is selected from a group consisting of halogen.

2. The method of claim 1, characterized in that the R₁, and R₂ are selected from the group consisting of Cl; and R₅ is selected from the group consisting of F.

3. The method of claim 1, characterized in that the R₃, and R₃′ are selected from the group consisting of hydrogen; and R₄, and R₄′ are respectively independently selected from the group consisting of hydrogen, —CH₃, —CH₂CH₃, and —CH═CH₂.

4. The method of claim 1, characterized in that the sulfite ester compound is selected from one of the following compounds or a combination of more than two of them:

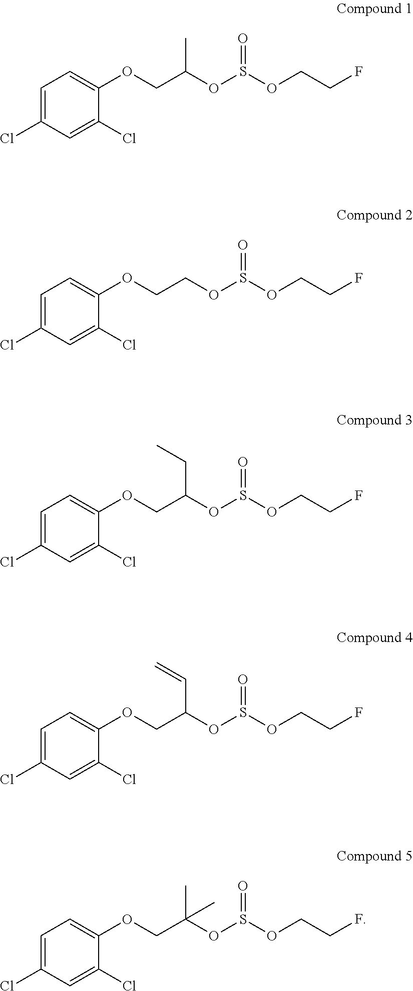 [SMILES: CC(COc1ccc(Cl)cc1Cl)OS(=O)OCCF ; O=S(OCCF)OCCOc1ccc(Cl)cc1Cl ; CCC(COc1ccc(Cl)cc1Cl)OS(=O)OCCF ; C=CC(COc1ccc(Cl)cc1Cl)OS(=O)OCCF ; CC(C)(COc1ccc(Cl)cc1Cl)OS(=O)OCCF]

5. The method of claim 1, characterized in that the sulfite ester compound is selected from one of the following compounds or a combination of more than two of them:

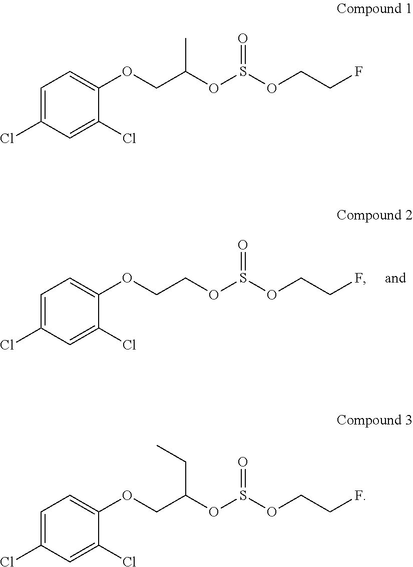 [SMILES: CC(COc1ccc(Cl)cc1Cl)OS(=O)OCCF ; O=S(OCCF)OCCOc1ccc(Cl)cc1Cl ; CCC(COc1ccc(Cl)cc1Cl)OS(=O)OCCF]

6. The method of claim 1, characterized in that the application concentration of the sulfite ester compound is not less than 0.001% (w/w).

7. The method of claim 3, characterized in that the application concentration of the sulfite ester compound is 0.001%-50% (w/w).

8. The method of claim 1, characterized in that when applied, it further comprises a surfactant and/or a solvent.

9. The method of claim 8, characterized in that when applied, the mass ratio of the sulfite ester compound to the surfactant is 1:0.1-20, and the concentration of the surfactant is not less than 0.001% (w/w); with the balance being solvent.

10. The method of claim 8, characterized in that the surfactant is selected from one of ethoxylated castor oils, sodium lauryl sulfates, saponins, ethoxylated alcohols, ethoxylated fatty esters, alkoxylated diols, ethoxylated fatty acids, carboxylated alcohols, carboxylic acids, fatty acids, ethoxylated alkylphenols, fatty esters, sodium dodecyl sulfide, and Tween or a combination of more than two of them; and
The solvent is selected from one of water, ethanol, isopropanol, benzyl alcohol, acetone, acetophenone, water, glycerol, castor oils, ethoxylated alcohols, ethoxylated amides, glycerides, butanol, 1-propanol, hexanol, other alcohols, dimethyl ether, and polyethylene glycol or a combination of more than two of them.

11. The method of claim 1, characterized in that the sulfite ester compound is prepared into an agricultural product, which further comprises one or more of an auxiliary dispersant, a wetting agent, a binder, an emulsifier, a stabilizer, and a solvent.

12. The method of claim 1, characterized in that the dosage form of the product is missible oil, water aqua, emulsion in water, soluble agent, microemulsion, suspension concentrate, oil suspension concentrate agent, microcapsule suspension concentrate agent, and mother liquor.

13. The method of claim 1, characterized in that the agricultural products made from the sulfite ester compounds are combined with foliar fertilizers, water-soluble fertilizers, compound fertilizers, pesticides, plant regulators, biostimulants, and soil conditioners.

14. The method of claim 1, characterized in that when in use, the sulfite ester compounds, or the agricultural products made therefrom are used for seed treatment, foliar application or root-irrigation.

15. An anti-evaporation agent, characterized in that the active ingredients of which comprise sulfite ester compounds, and the structural formula of the sulfite ester compounds is as follows: 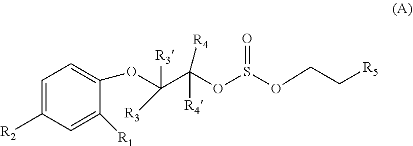
Wherein, R₁, and R₂ are selected from the group consisting of halogen;
R₃, R₃′, R₄, and R₄′ are respectively independently selected from the group consisting of hydrogen, C₁-C₅ alkyl, and C₁-C₅ alkenyl; and
R₅ is selected from a group consisting of halogen.
